(19) United States
(12) Patent Application Publication
(10) Pub. No.: US 2023/0016640 A1
(43) Pub. Date: Jan. 19, 2023

(54) SYSTEM AND METHOD FOR AUTOMATED AMBIENT MOBILITY TESTING

(71) Applicant: Chirp Inc., Waterloo (CA)

(72) Inventor: Parthipan SIVA, Waterloo (CA)

(21) Appl. No.: 17/846,547

(22) Filed: Jun. 22, 2022

Related U.S. Application Data
(60) Provisional application No. 63/221,065, filed on Jul. 13, 2021.
(60) Provisional application No. 63/221,074, filed on Jul. 13, 2021.

(51) Int. Cl. A61B 5/11; G16H 50/70

(52) U.S. Cl. CPC A61B 5/112; A61B 5/1126; G16H 50/70; A61B 2562/0271

(57) ABSTRACT

A system for ambient mobility testing including: at least one sensor configured to collect data associated with an individual's movement; an analysis module configured to analyze the collected data to determine a set of timed-up-and-go (TUG) actions of a TUG test and determine results of a complete TUG test; and a reporting module configured to provide the results of the TUG test. A method for ambient mobility testing, the method including: collecting data associated with an individual's movement, via at least one sensor; analyzing the collected data to determine timed-up-and-go (TUG) actions of a TUG test; determining results of a complete TUG test; and providing the results of the TUG test.

### RELATED APPLICATIONS

The present disclosure claims the benefit of U.S. Provisional Application No. 63/221,065, filed Jul. 13, 2021, and of U.S. Provisional Application No. 63/221,074, filed Jul. 13, 2021, both of which are incorporated herein in their entirety.

### FIELD

The present disclosure relates generally to a system and method for automated ambient mobility testing. More particularly, the present disclosure relates to a system and method for automated ambient mobility testing, and in particular for timed-up-and-go (TUG) testing using ambient sensors.

### BACKGROUND

When a person falls, there can often be various complications, particularly for older individuals. For example, falls remain a leading cause of injury-related hospitalizations among older people and/or seniors, and it has been reported that between 20% and 30% of seniors fall each year. Falls and associated outcomes may not only harm the individuals but are shown to also affect family, friends, care providers, the health care system, and the like. With an aging population, this cost is expected to continue to increase. Detecting fall risk is an important component of a geriatric assessment because effective interventions to prevent future falls and limit their consequences are available. In the absence of an actual injury from a fall, many risks (including previous falls) are seldom reported to the primary care physician.

Based on American Geriatrics Society/British Geriatric Society Clinical Practice Guideline for Prevention of Falls in Older Persons seniors at risk of fall or those who suffer from mobility decline may undergo functional assessment (gait balance tests). Commonly used tests include: Get up and go test, Timed up and go test (variation of get up and go test), Berg Balance Scale, and Performance Oriented Mobility Assessment. Based on CDC guidelines, functional assessment tests are suggested including Timed up and go (TUG) which has an optional 30 second chair stand and a 4-stage balance test. Other jurisdictions also recommend this or similar tests. Additional gait/balance metrics are also possible for an assessment.

Functional assessment metrics are designed to be observed or tested in a clinical setting with the aid of props. The duration of the tests may vary, however the shorter tests with little or no props (for example TUG tests) are preferred for clinical settings. Due to its simplicity, TUG is recommended by both CDC and UK NHS. The TUG test includes, for example, observing and timing participants while they rise from an armed chair of approximately 46 cm seat height and 65 cm arm height, walk at their usual pace a distance of 3 m towards a line marked on the floor, turn 180 degrees, walk back to the chair, and sit down. In some cases, the participants may be asked to wear their regular footwear and use any customary walking. The time taken to complete the test is measured by a stopwatch and a faster time indicates a better performance.

CDC recommends a TUG cutoff of 12 seconds (sec). More than 12 seconds indicates an individual is at a greater risk of fall. While a mobility assessment test like TUG is used as part of geriatric assessment, it is conducted in a clinical setting during annual checkups. In order to increase the frequency of mobility assessment and to remove biases in observer timing of the TUG sequence automating the TUG test may be preferred. As these tests tend to be conducted in a clinical setting, the tests may not be frequent enough to determine an individual's risk of falling.

In order to provide true continuous monitoring of mobility in a home there is a need for a system for mobility testing that overcomes at least some issues with conventional systems, is privacy preserving and does not require users to change their normal daily routines.

### SUMMARY

According to an aspect herein, there is provided a system for ambient mobility testing including: at least one sensor configured to collect data associated with an individual's movement; an analysis module configured to analyze the collected data to determine a set of timed-up-and-go (TUG) actions of a TUG test and determine results of a complete TUG test; and a reporting module configured to provide the results of the TUG test.

In some cases, the at least one sensor may be a privacy preserving sensor.

In some cases, the at least one sensor may be an mm-wave radar, LIDAR sensor or a WI-FI sensor.

In some cases, the system may further include a monitoring module configured to monitor for TUG actions to update the results of the TUG test.

In some cases, the analysis module may be further configured to determine a decline in mobility from the TUG actions.

In some cases, the analysis module may be further configured to determine an abnormality within the results of the TUG test or within the TUG actions.

In some cases, the at least one sensor may be configured to commence monitoring the individual's movement on a start command.

In some case, the at least one sensor may be configured to continuously monitor and the analysis module is configured to determine which collected data is associated with a TUG action.

In some cases, the analysis module may be configured to aggregate a plurality of TUG actions performed during a predetermined period of time to determine a set of actions for the TUG test.

In some cases, the predetermined period of time may be between 1 hour and 1 week.

In another aspect, there is provided a method for ambient mobility testing, the method including: collecting data associated with an individual's movement, via at least one sensor; analyzing the collected data to determine timed-up-and-go (TUG) actions of a TUG test; determining results of a complete TUG test; and providing the results of the TUG test.

In some cases, the data may be collected via a privacy preserving sensor.

In some cases, the at least one sensor may be an mm-wave radar, LIDAR sensor or a WI-FI sensor.

In some cases, the method may further include monitoring for TUG actions to update the results of the TUG test.

In some cases, the method may further include determining a decline in mobility from the TUG actions based on the results of the TUG test.

In some cases, the method may further include determining an abnormality within the results of the TUG test or within the TUG actions.

In some cases, the collecting of data may commence on a start command.

In some cases, the data may be continuously collected and analyzed to determine which collected data is associated with a TUG action.

In some cases, the method may further include aggregating a plurality of TUG actions performed during a predetermined period of time to determine a set of actions for the TUG test.

In some cases, the predetermined period of time may be between 1 hour and 1 week.

### BRIEF DESCRIPTION OF FIGURES

Other aspects and features of the embodiments of the system and method will become apparent to those ordinarily skilled in the art upon review of the following description of specific embodiments in conjunction with the accompanying figures.

Embodiments of the system and method will now be described, by way of example only, with reference to the attached Figures, wherein:

FIG. 1 illustrates an environment for a Timed Up and Go (TUG) test;

FIG. 2 is a flow chart that illustrates a method of performing a TUG test using the environment of FIG. 1;

FIG. 3 is system for automated ambient testing according to an embodiment;

FIG. 4 is a flow chart that illustrates a method of performing a TUG test using the environment of FIG. 1 in an automatic setting;

FIG. 5 illustrates a system for automating the standard TUG test in a home setting according to an embodiment;

FIG. 6 is a flow chart that illustrates a method of performing a TUG test in a passive piecewise setting;

FIG. 7 illustrates a system for passive piecewise estimation of TUG metrics in a home setting;

FIG. 8 illustrates a method for automatically detecting TUG and estimating of passive piecewise timed up and go (P2TUG) according to an embodiment;

FIG. 9 illustrates a method for radar signal processing and may be employed in the method of FIG. 8 according to an embodiment;

FIG. 10 illustrates a method for tracking according to an embodiment;

FIG. 11 illustrates a method for action detection according to an embodiment; and

FIG. 12 illustrates a method for TUG detection according to an embodiment.

### DETAILED DESCRIPTION

In the following, various example systems and methods will be described herein to provide example embodiment(s). It will be understood that no embodiment described below is intended to limit any claimed invention. The claims are not limited to systems, apparatuses or methods having all of the features of any one embodiment or to features common to multiple or all of the embodiments described herein. A claim may include features taken from any embodiment as would be understood by one of skill in the art. The applicants, inventors or owners reserve all rights that they may have in any invention disclosed herein, for example the right to claim such an invention in a continuing or divisional application and do not intend to abandon, disclaim or dedicate to the public any such invention by its disclosure in this document.

Generally, the present disclosure provides a system and method for automated ambient mobility testing. The system is intended to sensors configured to detect particular mobility movements in order to determine whether a test or a test sequence is being initiated. The system is figure configured to review the sequence and determine testing metrics. The system further includes the ability to provide testing results or anomaly detection in order to determine potential mobility decline.

Existing and conventional automated Timed Up and Go (TUG) tests can be generally categorized by sensor types and whether the approach is supervised or unsupervised.

There have been several conventional methods based on video using pose detection and action recognition. While video is an ambient sensor, it may not be privacy preserving for use in sensitive areas like bedrooms. Further, it may require calibration for measuring distance travelled. Some methods may go beyond the traditional visible light cameras and use depth cameras, which are more privacy preserving and can measure distances but are limited in the area they can monitor.

Generally, each individual action component of TUG (stand up, walk, turn, walk, turn, sit-down) is detected using action detection techniques and is timed. Gait speed during walking is also measured. The combined action times is provided as the TUG metric with additional breakdown of times and gait metrics (for example, speed, cadence, and the like).

As a deviation from standard TUG, conventional video-based method may look only at gait metrics during walking and may correlate those results to TUG results. This can result in a TUG estimation based only on walking observed in the video, which may ignore lower extremity strength needed for sitting and standing and may result in inaccurate estimations.

Other conventional solutions may use wearable sensors. Wearable sensor approaches may be based on Inertial Measurement Unit (IMU) sensors (which may include, accelerometer and gyroscope). Some methods may further use an individual's smartphone, with its integrated accelerometer and gyroscope, instead of instrumenting people with an IMU sensor. Some smartphone approaches may augment IMU with additional Bluetooth based non-wearable sensors (for example a pressure sensor or the like) that can be connected to the smartphone. Using the IMU sensor and additional sensor data each component action of TUG (stand up, walk, turn, walk, turn, sit-down) is intended to be detected and timed. The combined time is presented as a TUG metric with additional breakdown of times and gait metrics (for example, speed, cadence, and the like)

It has been determined that fewer ambient approaches, excluding video cameras, exist in comparison with the other approaches. These methods tend to be primarily based on instrumenting chairs and/or a walk area. Distance travelled is determined using light barriers and or Infrared [IR] sensors. Since a plurality of sensors are typically used, each sensor type may indicate a different component of the TUG test. In these cases, no detection/classification of sensor data may be needed. These methods generally provide TUG time and limited gait metrics in comparison to wearable and video-based systems.

For TUG tests, a supervised/unsupervised test is used to refer to if another person is supervising the test or not. Unsupervised approaches are useful for in-home continuous assessment. Supervised approaches rely on a supervisor to start the TUG test and validate that the patient properly executed the test while the system does the timing. With a system doing the time, it is intended to remove supervisor bias in timing and additional gait metrics.

Few unsupervised approaches exist. The approaches are generally based on a wearable IMU sensor by itself or in addition to instrumented chairs. These conventional methods tend to be the most hands-off unsupervised approach. In general, the conventional method may use IMU data to estimate TUG test results based on the detection of TUG like sequence of actions (stand up, walk, turn, walk, turn, and sit-down). If a TUG test like sequence is performed during the daily activity it can be detected and timed. Based on the unsupervised TUG estimation looking for TUG like sequences in normal daily activity resulted in slower TUG times. Self-selected walking pace during normal daily activity may be slower than a supervised TUG test situation. In the context of TUG test, supervised TUG tests may be referred to as active and unsupervised as passive to avoid any confusion between supervised learning used in machine learning

There have been several limitations of the conventional solutions that have been observed. In particular, most automated systems look at automating the timing of an administrated TUG test in a clinical setting and as such may not be useful for continuous monitoring of mobility on a daily basis. Systems that can be self-administered can be done more frequently. However, this again requires the user to self-administer the test on a regular basis without forgetting. A complete passive system without user effort may address some of these issues.

In some cases, wearable devices have shown some promise for passive monitoring of mobility by detection of TUG like sequences. However, these solutions require users to wear a sensor and will likely require charging the device regularly. The devices are not guaranteed to be with the users at all times. Non-wearables devices for passive monitoring may be preferable because these devices would not require users to change their behavior and remember a wearable device.

While camera-based systems present a simple single non-wearable ambient device solution; they do not tend to be privacy preserving for use at home. Depth cameras can preserve privacy; however, they generally have very limited field of view.

There have been several attempts at automating the TUG test. The most relevant approaches have been active methods, video-based methods and IMU methods. In particular, active methods may use a plurality of sensors (force sensor, optical distance sensor, or the like) affixed to chair and surrounding areas to measure a TUG test that is administrated by a clinician. This method is intended to involve a supervisor who instructs the subject to perform the test with a plurality of sensor instrumentation in the room.

Video based methods require calibration and are generally not privacy preserving. Furthermore, most of these methods have not been able to estimate mobility (e.g., via TUG) passively during normal daily activities. A conventional approach may be able to passively estimate mobility parameters from video-based gait analysis during walking and uses regression to attempt to map these characteristics to TUG score.

Further, IMU methods require a test subject to be instrumented with a sensor. Some also require additional props such as a TUG chair. Furthermore, few of these methods estimate TUG passively during normal daily activity. These methods do not look for subcomponents of TUG test sequence separately throughout the day and combine them together for mobility metrics.

As such, it has been observed that the conventional methods may not provide accurate results for daily activity. Further, with the requirements of having individual wear sensors, or otherwise change their daily routine, the TUG results may be lacking or incorrect. Embodiments of the method and system detailed herein are intended to provide accurate TUG results without having an individual outfitted with additional equipment or having a further individual monitor the test. In particular, embodiments of the system and method are intended to provide for ambient testing to detect and determine the likelihood an individual may be suffering from mobility issues.

Embodiments of the system and method are intended to provide for passively and continuously assessing the mobility of individuals during their normal activities in the home. Furthermore, embodiments of the system and method are intended to provide an interactive method for direct automated testing of mobility that can be self-administered or administrated by a clinician or caregiver. Direct testing of mobility may be accomplished by automating the standard timed up and go (TUG) test and the continuous passive assessment is accomplished by estimating TUG test times from passively observing normal daily activities (for example, sit down, stand up, walk, turn) in the home. Further, embodiments of the system and method are intended to track mobility parameters over time to identify anomalies or declines in mobility.

FIG. 1 illustrates an environment for a system 100 for automating ambient testing and, in particular, the standard timed up and go (TUG) test. The system 100 includes at least one ambient sensor 105. The ambient sensor 105 is configured to automatically detect and measure components and/or actions (for example, stand up, walk, turn, walk, turn, sit-down) of a TUG test. The system 100 may automatically detect and determine each action and analyze the time to complete each action. Furthermore, the velocities and location of body limbs during the action may also be determined. For a walking action, additional gait metrics (for example, step stride, posture, speed) may also be measured. The total time to complete the entire TUG sequence may be determined or, in the case of passive operation, may be estimated.

The system 100 may be operated in a manual/interactive setting, an automatic setting, and/or a passive piecewise setting. The system 100 may track TUG test times over a prolonged period of time to detect declines or deviation from the acceptable cutoffs. In some cases, a prolonged period of time may be in the range of 1 month, 1 year, 2 years or the like. In a manual/interactive setting, a supervisor (for example, health care professionals, caregiver, or the like) or the test subject themselves may set up a chair and a space to walk 3 m, as shown in FIG. 1. At least one ambient sensor 105 may be placed with a field of view of the entire 3 m distance and chair. The ambient sensor 105 may be placed anywhere on a hemisphere. The chair, 3 m walkway and the person are preferably entirely within the sensor device's field of view (FOV) at all times during the TUG test. Ideal sensor placement is behind the chair facing down the walkway or at the end of the walkway facing the chair, as shown in FIG. 1.

Different sensors have different fields of view and different sensitivity. Generally, sensors placed on the hemisphere will have a full view of the TUG sequence. In embodiments of the system and method detailed herein, at least one sensor placed within the hemisphere will provide TUG test results, but a preferred location of at least one sensor may be behind a chair in order to provide the most accurate measurement of distance travelled and detection of stand/sit movements. If the at least one sensor is placed, for example, at an end of a walkway, the sensor may provide similar accuracy in distance traveled but slightly lower accuracy for stand/sit compared to behind chair.

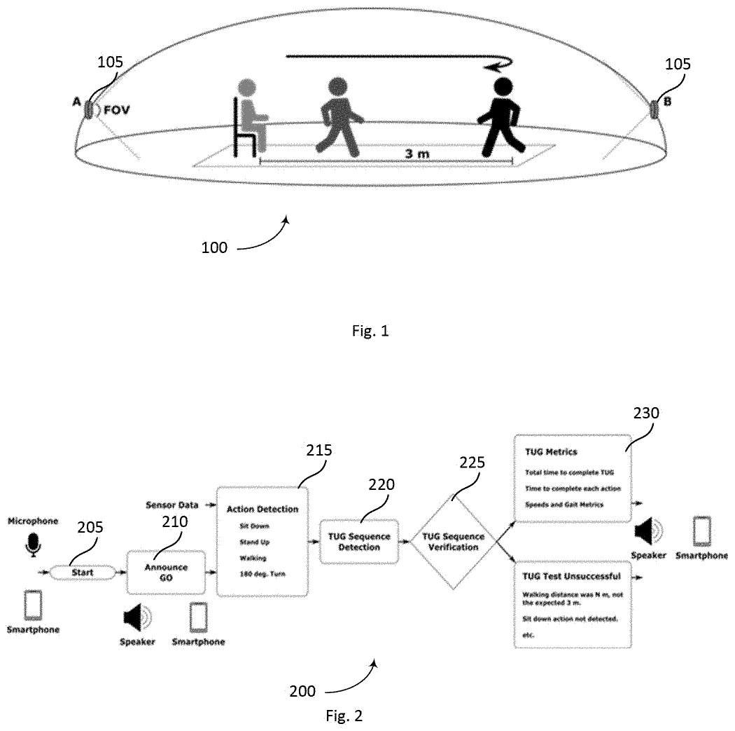

FIG. 2 is a flow chart that illustrates a method 200 of performing a TUG test using the system 100, in this case, in a manual/interactive setting. Having a test subject in a seated position the test subject or supervisor activates the system 100 for a TUG test, for example, via a voice interface, a smartphone interface, or the like, at 205. Once activated, the system indicates “go” through an audible sound, at 210, at which point the test subject performs the TUG sequence (stand up, walk 3 m, turn around, walk back and sit down), at 215. The system 100 detects the TUG sequence, at 220 and may confirm or otherwise indicate the TUG test was successful or unsuccessful, at 225. A test may be considered unsuccessful if various test parameters were not met, for example, if person did not walk 3 m, if a sit, down action was not detected, or the like. The system 100 is configured to indicate to the test subject or third party that the test was unsuccessful and which part of the test was unsuccessful. In some cases, this may be received via audio of the system or a mobile phone or other computer interface. On successful completion of the test, TUG metrics may be reported to a test subject, a health care professional or a third party and may further be retained for trend tracking, at 230.

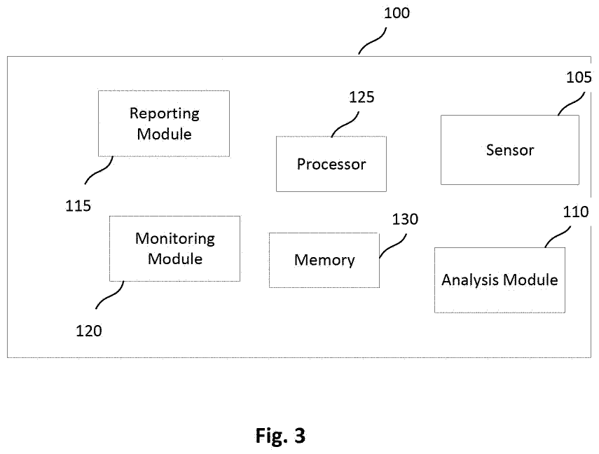

FIG. 3 illustrates the system 100 for ambient TUG testing. The system 100 includes at least one ambient sensor 105, an analysis module 110, a reporting module 115, a monitoring module 120, at least one processor 125 and at least one memory component 130. The system is generally intended to operate within a device connected to a network, such as WI-FI, Bluetooth, or the like but may be distributed over various devices. The modules, including the processor 125 and memory 130, are in communication with each other but may be distributed over various devices or may be housed within a single device. The processor may be configured to retrieve stored instructions from the memory 130 and execute the instructions that provide for the functionality of the modules.

The at least one sensor 105 is configured to sense movement of a test subject within an environment. The at least one sensor 105 is intended to be placed in the environment and sense an individual's movement in the environment without needing physical contact with the individual. The at least one sensor 105 is intended to be configured with a large enough field of view to view an entire TUG sequence (stand, walk, turn around, walk back, sit) or monitor an entire room in a home setting. The at least one sensor 105 may be configured to measure movement in vertical axis (for detecting sit, stand) as well as horizontal movement to detect walking. Further, the at least one sensor is intended to avoid video, photographic or detailed scan which may be an invasion of privacy. In some cases, the at least one sensor 105 may also be configured to detect movements of the individual's limbs, this is intended to allow the system to provide for additional gait metrics but may not be required for basic TUG estimation. In some cases, the at least one sensor may be a radar, WI-FI sensor, LIDAR sensor, low-resolution thermal sensor or the like.

The analysis module 110 is configured to receive sensor data from the at least one sensor 105 and determine whether the data is sufficiently detailed to consider a recently performed TUG test successful. If so, the analysis module 110 is configured to analyze the data and determine the TUG test results as detailed herein.

The reporting module 115 is configured to receive or retrieve the analyzed data from the analysis module 110 and report the test results. In some cases, the reporting module 115 may provide a report, which includes the time to perform an entire TUG sequence. In other cases, the reporting module 115 may include time to perform each action in the TUG sequence as well as the total time. In some cases, the reporting module 115 may further include metrics on the walking portion of the sequence, including aspects such as, walking speed, step size, body posture, and the like.

The monitoring module 120 is configured to receive and review sensor data during passive monitoring. In some cases, the monitoring module 120 may provide reviewed data to the analysis module 110 to be used in determining TUG results.

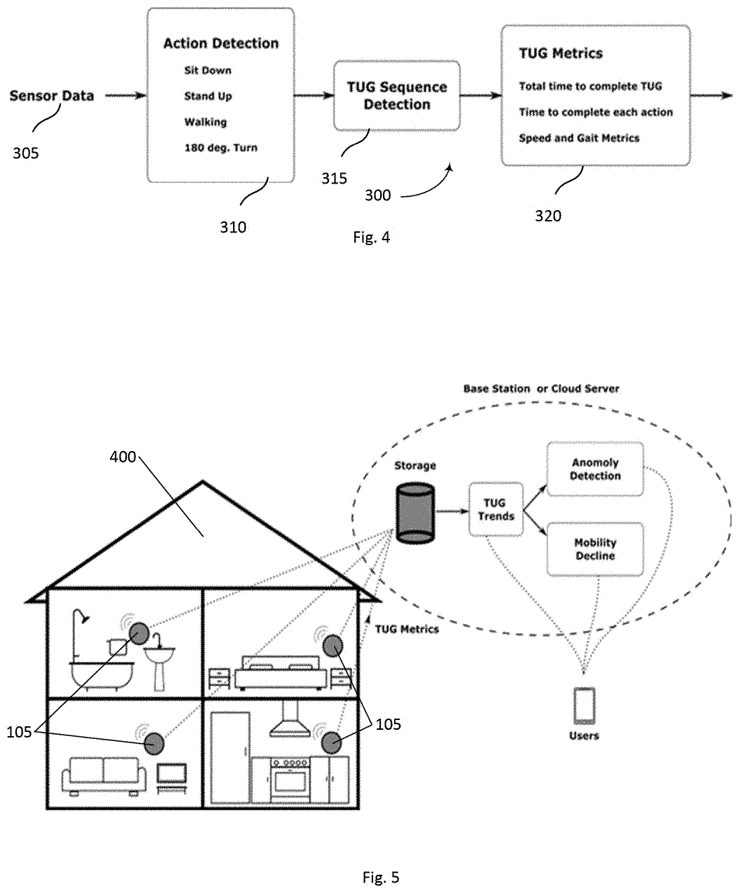

FIG. 4 is a flow chart that illustrates a method 300 of performing a TUG test using an embodiment of the system 100 in an automatic setting. A supervisor (for example, health care professionals, caregivers, or the like) or the test subject may setup the system 100, at 305. In some cases, setup may include plugin in the sensor device to power. If a mobile phone, smartphone or other interface is to be used the sensor may be pair to that device and communication settings may be configured (for example, via Bluetooth, WI-FI, or the like). If processing is to happen outside the device (for example, within a base station, cloud environment or the like) communication to the external processing may also be established during setup. must be established in the same way it was done to smartphone etc. Physical object, such as a chair and a 3 m walkway may also be setup or may have previously been configured for the TUG test.

The supervisor or test subject may be able to start the TUG test procedure at any point without interacting with the system 100. Once the at least one sensor 105 is powered up and connected to a desired interface and/or base station, it is intended that the at least one sensor may automatically go into monitoring mode and look for TUG sequences. No further interaction is intended to be needed. All detected tests may be sent to a smartphone or device that is paired to the system. In some cases, the system 100 may be continuously and passively monitoring the test subject and may detect when a TUG sequence occurs in its field of view, at 310. At 315, the system 100 automatically detects each action of the TUG sequence and recognizes when a complete TUG sequence (which may include stand up, walk 3 m, turn around, walk back and sit down) has occurred. The TUG metrics, when a complete TUG sequence is detected, may be analyzed and reported to user and/or retained for trend tracking, at 320.

FIG. 5 illustrates a system 100 for automating an ambient TUG test system in a home setting. The system 100 may include a plurality of ambient sensors 105 spread across the home 400, and each sensor may be configured to detect TUG steps or sequence. The sensors 105 may continuously scan for TUG sequence to occur, for example, each sensor may be continuously operating scanning for test subject movement. The system 100 aggregates TUG data from either a single or a plurality of ambient sensors 105 to a central processing location, to be reviewed and analyzed by the system. In some cases, the at least one sensor 105 may be connected to and send data to a base station or cloud server), where further modules of the may system reside. The system 100, including the analysis module and monitoring module may perform post-processes with the data to determine mobility trends. When a TUG sequence is detected, TUG metrics as well as detailed timing and speed metrics may be sent to a central processing station, for example the processor 125. The monitoring module in conjunction with the central processing station is configured to detect trends and anomalies in the TUG metric over time. The data used to detect trends may be stored in the memory module 130, for example a database. Trends may be used to automatically detect and flag any anomalies and/or declines in TUG measurements.

TUG actions can be detected via, for example, machine learning (any classifier such as rule based, support vector machine, neural networks or the like,) trained on collected sensor data to detect each action. In some cases, various tracking modules, for example, Kalman Filter, Particle Filter, Mean Shift, data association, or the like, may be used to identify walking speed and other metrics. Sensor data are collected as a time sequence (for example: 10 Frames Per Second (FPS), or at another frame rate per second). The start and end of the action in the sensor data is identified and timed by looking at a time stamp provided by the sensor or using the number of frames spanning the action and the frame rate of the sensor.

In some cases, decline may be measured as above a predetermined cut off time (for example, 12 sec per CDC. In other cases, decline may be determined for example, via a slope of a linear fit to TUG time or the like. Anomaly may be detected as a large deviation from a trend line. For example, a linear fit to TUG time followed by 2 standard deviations from the fit line may be considered an anomaly. Other statistical tests can also be used. Similar trend fits can be done on individual TUG action times and walking metrics for detailed trend tracking and anomaly detection.

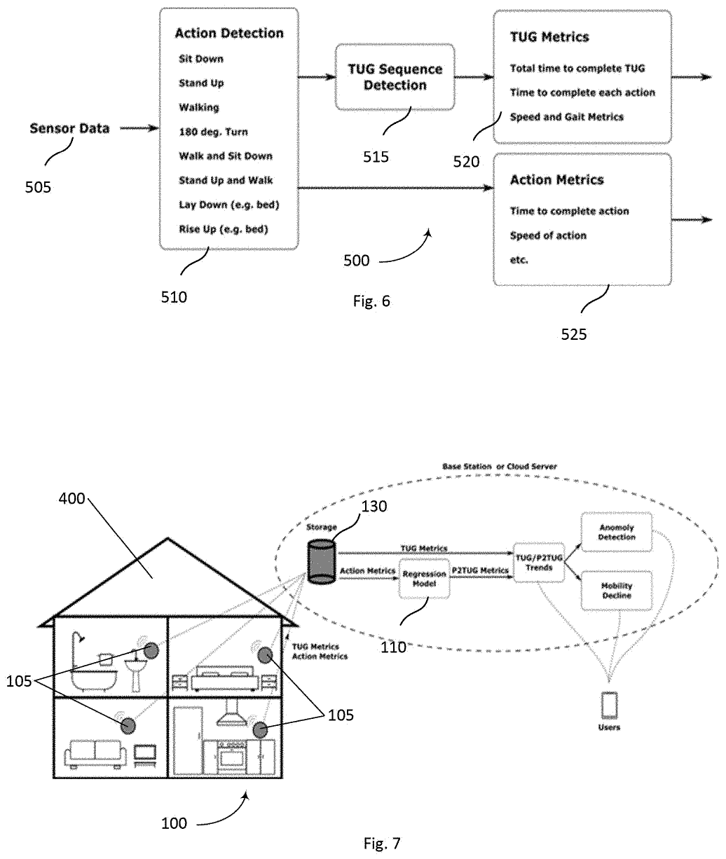

FIG. 6 is a flow chart that illustrates a method 500 of performing a TUG test using in a passive piecewise setting. At 505, a subject is intended to perform the TUG sequence (or the TUG sequence otherwise occurs) as part of daily activity. In some cases, at 510, other actions may be detected and analyzed by the system. In some cases, the sensors may continuously monitor activity or monitoring mobility at various times throughout the day. Relying on the subject to perform TUG sequence (either on their own or with alerts) may require compliance and effort on the subject's part, while the natural occurrence of TUG sequence in daily activity is likely to be rare, at 515. To accommodate a lack of subject compliance, a system 100 may be set up in a subject's home, and method 500 may be employed using a plurality of ambient sensors 105 spread through the home to detect and measure the actions involved in TUG as well as subsets of these action that occur consecutively, at 520. The ambient sensors 105 of the system 100 are intended to determine occurrences of TUG sequence and report individual action metrics (for example, time for completion of sit-down, or the like). Using the measured actions and subsets of actions, at 525, a regression model may be used to estimate TUG, referred to hereinafter as passive piecewise timed up and go (P2TUG).

In some cases, a sample population to measure TUG action times may be used, then a feature may be determined, for example, sum of all median TUG action, subsets of actions or the like. This may be followed by a linear regression to clinically administrated TUG time. Alternatively, other regression models can be trained from a sample population including Neural Networks (NN) that are configured to learn the feature and the regression model at the same time.

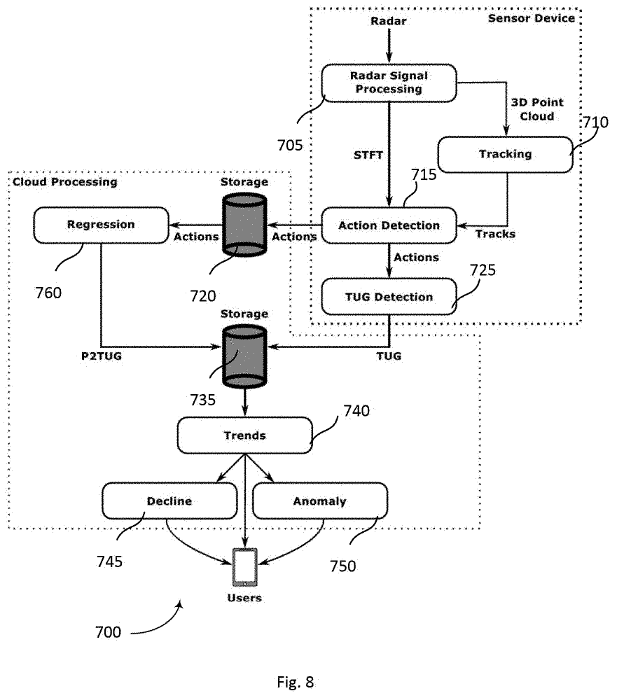

FIG. 7 illustrates the system 100 used for passive piecewise estimation of TUG metrics in a home setting 400. At least one sensor 105 is placed in at least one room of the home 400. The at least one sensor 105 may continuously or periodically scan for TUG sequence of movements and actions related to TUG. TUG metrics as well as individual TUG action metrics are sent to the analysis module 110 to be reviewed. A central processing station, for example the at least one processor 125 may perform and execute actions while the memory component 130 stores both TUG and action metrics over time. In some cases, at least some components of the system 100 may be accessed via a base station or cloud server. Action metrics over a span of time may be converted to an estimate of TUG metric using a regression model, using, for example, P2TUG. TUG and P2TUG results are intended to be combined by the system to form trends and detect anomalies. The component actions of the TUG that may be independently detected and timed for estimating P2TUG include, for example: sit down; stand up; walking followed by sit down; stand up followed by walking; walking; 180 degree turns; walking followed by and/or preceded by 180 degree turns or the like.

In an attempt to capture the mobility of user in the home, some additional actions may also be detected and timed and may further be used in the regression model. These additional actions include, for example, time to lay down (e.g., get into bed) time to raise up (e.g., get out of bed) and the like. As detailed herein, TUG and/or P2TUG data may be analyzed for trends and anomalies, by for example, the analysis module 110. TUG/P2TUG data from single or a plurality of sensors 105, in a home setting may be aggregated in a central location (for example, the system, via a base station, cloud server, or a single device) and post-processed to determine trends. Trends are intended to be used to automatically detect and flag any anomalies and/or declines in TUG measurements.

In select embodiments, the at least one sensor 105 may be, for example, a mm-wave radar which may include a 60-64 GHz Frequency Modulated Continuous Wave (FMCW) radar. The sensor may have a 120-degree azimuth and elevation field of view. The sensor may further include a microphone, a speaker, Wi-Fi, a micro-processor, and the like. The mm-wave radar is intended to preserve privacy, for example by tracking people and the actions they perform, without personal information. In this way, actions (such as, sit down, stand up, walking 180 degree turns and the like) that occur during a TUG test can be detected without a wearable device. The microphone and speakers may be used for performing an interactive TUG test. The micro-processor may run Linux, or similar system, and may be used for signal processing and detection. Wi-Fi may be used to transmit the TUG metrics and TUG action metrics to a cloud processor for TUG time estimations and trend tracking. It will be understood that other network transmissions may be used.

FIG. 8 illustrates a method 700 for automatically detecting TUG and estimating of P2TUG according to an embodiment. The input to method is provided by one or more ambient sensors 105, at 705. The signals are processed and may be tracked at 710. The test subject actions are detected, at 715 and may be saved in storage at 720. The actions are reviewed to determine TUG detection, at 725. The actions may be analyzed and processed by the analysis module via regression models at 730. The results of TUG actions and the analyzed action may be stored in the memory component at 735. At 740, the analysis module may review the data for trends. The analysis module 110, may further review the data for a decline in mobility, at 745 or any anomaly, at 750. Any results may then be reported to a user or to a medical professional or the like. It is intended that the analysis module may review and determine various TUG actions over a predetermined period of time, for example, an hour, a day, two days, a week, or the like, and may aggregate these various actions performed over the predetermined period of time to determine a complete set of TUG actions.

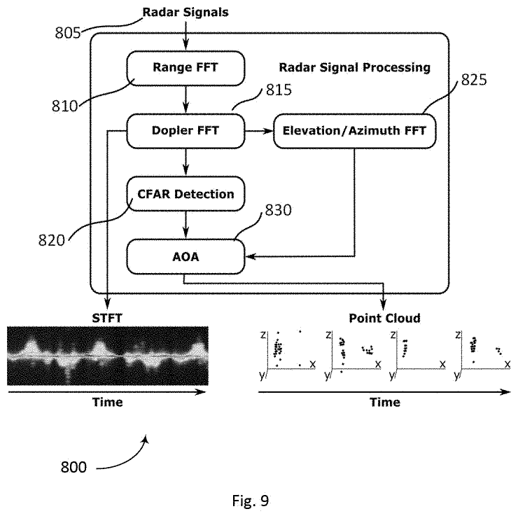

FIG. 9 illustrates a method 800 for radar signal processing, which may be employed in method 700. As outlined in FIG. 9, radar signals may be received at 805 and may include range Fast Fourier Transform (FFT) analysis, at 810, and Doppler FFT analysis, at 815, may be performed to obtain the range Doppler map. The range Doppler map may be used by the constant false alarm rate (CFAR) method, at 820, to detect objects in the scene. Static objects are eliminated by removing DC component from the range Doppler map before CFAR detection. Using azimuth and elevation FFT, at 825, the angle of arrival (AOA) of the detected objects are computed to produce a 3D point cloud of objects in the scene, at 830. Additionally, the range Doppler map may be integrated along the range dimension and used as a short time Fourier transform (STFT) of velocity profile. Both STFT and 3D point cloud may be used for action detection and tracking. In other words, the raw data is transformed into a 3D point cloud and a short time fast Fourier transform of velocity profiles.

In some cases, alternative methods may also be possible. In some cases, methods may work right on the radar cube (for example, Doppler Map time series) to do action detection and tracking. Other methods may use raw radar signals.

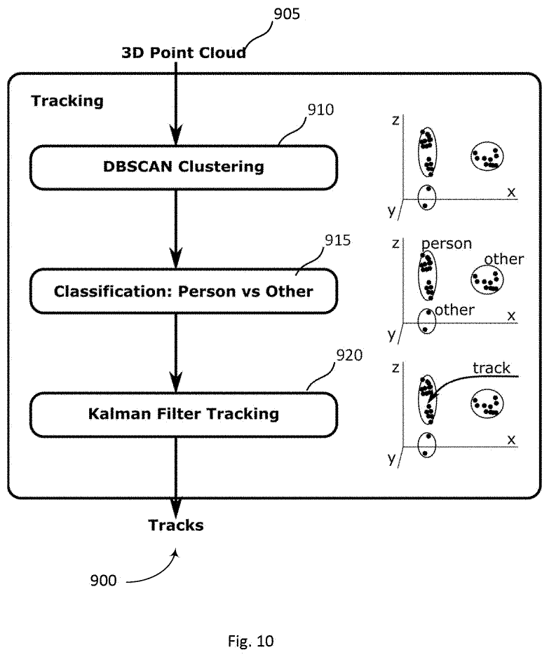

After the radar signals are processed, the movement of individuals within the signals may be tracked. FIG. 10 illustrates a method 900 for tracking data signals and may be employed in method 700. A tracking by detection peridium is used on the 3D point cloud, at 905, to detect and track people in the scene that was detected by the sensor. Detection may be accomplished by Density-based spatial clustering of applications with noise (DBSCAN) clustering, at 910. Clusters are represented using features such as location, number of points in cluster, speed of points in cluster, signal strength of points, relative distance of neighboring clusters, and the like. At 915, clusters are then classified as people or other using a shallow fully connected neural network. To preserve temporal consistency and to track people who are temporally stationary a constant velocity Kalman Filter may be used to track the people detection over time, at 920. In other words, tracking is performed via people detection and tracking in 3D point cloud. In some cases, instead of Kalman Filtering, the system may be configured to employ, Particle Filtering, Multiple Hypothesis Tracking or the like. Multiple Hypothesis tracking may provide accurate results but may not be able to work in real-time with very low latency.

In some cases, a different approach can also be used instead of tracking by detection. Example mean shift tracker can be used on the 3D point cloud without doing the clustering and detection. Other approaches may also be used as are known in the art and would be understood.

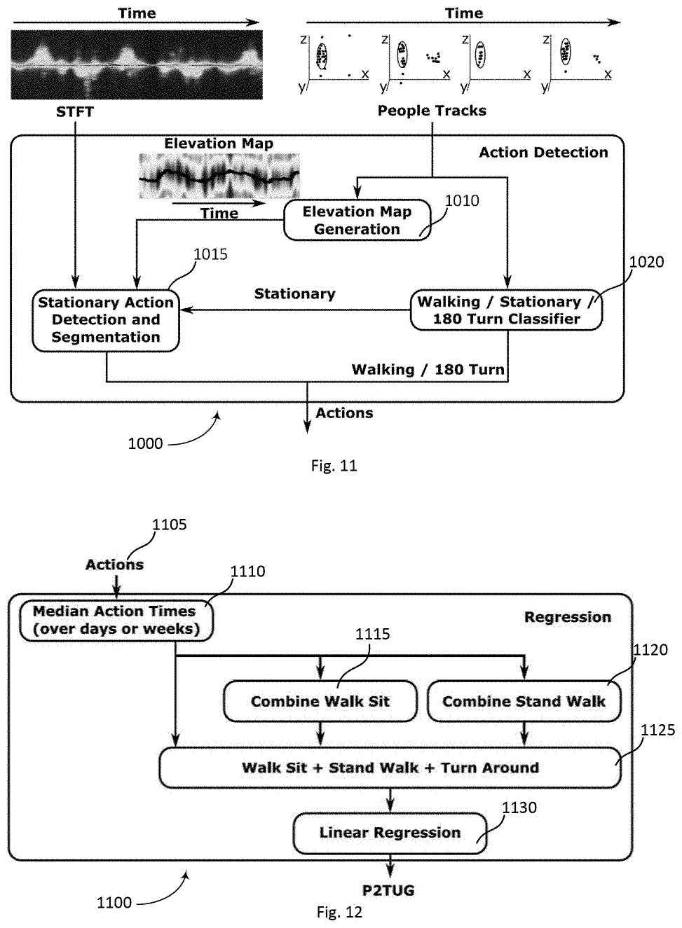

FIG. 11 illustrates a method 1000 for action detection and may be employed in method 700. At 1020, tracks are processed via a rule-based method to classify people as stationary, walking or turning 180 degrees. At 1010, people tracks are converted to an elevation map by a kernel density estimation in the elevation axis. The projection is intended to allow the elevation map to be processed by convolutional neural nets (CNN) action classifier in a similar manner as the STFT is processed. Elevation changes are important for sit down, stand up, lay down, and the like. Elevation map and STFT is used by a shallow CNN on all tracks identified by 1020 as stationary to further classify the track as a stationary action (for example, sit down, stand up, and the like) and refine the start and end of the stationary action, at 1015.

In some cases, the rules may be defined as follows: stationary—position has not changed and/or near zero velocity; walking—position is changing in a single direction and there is a consistent velocity vector pointed in the direction of motion; and 180 turn—180 degree change in direction of movement maintained over a time period (for example, 1 second, 2 seconds or the like).

FIG. 12 illustrates a method 1100 for TUG detection and may be employed in method 700. Results from the method for action detection may be stored in a memory component and may be reviewed by the monitoring module 125 or analysis module 110, at 1105. Each action may be detected, and the specific sequence of action needed for TUG test may be determined. Further, verification of the sequence may be accomplished in the form of verifying that test subject walked 3 m each way, started in a seated position and ended back at the start in a seated position. The time to complete an entire TUG test sequence as well as breakdown of each action times and walking speed are computed.

To determine P2TUG, P2TUG regression may be performed by the analysis module 110. Each action (sit, stand, walk, and the like) may be independently detected and stored, as detailed herein. Using these stored independent actions P2TUG may be estimated. Embodiments of the system and method are intended to obtain robustness for each action timing, so actions may be collected over a period of time, for example, hourly, daily, weekly or the like. At 1110, the median time to complete each action is computed. Alternative robust estimations, for example, average, outlier rejection methods or the like may also be used.

Further, walk-sit actions, at 1115, and stand-walk actions, at 1120, that had at least 3 m walking may be used to estimate the combined walk-sit and stand-walk actions. However, in the absence of any sit and stand events with 3 m walking before and after the action, a time to walk 3 m may be estimated from the observed median walking speed and combined with sit and stand times. Finally, a time estimate for a TUG like sequence (Stand-Walk, Turn Around, Walk-Sit) may be determined as the sum of all individual median times, at 1125. This result is intended to provide a daily or weekly accumulated median estimate. To get a more robust estimate of the TUG test a linear regression model may be used to map this piecewise accumulated median estimate to a more robust P2TUG estimate, at 1130. The linear regression model may be trained with data gathered from a test group who have been observed in the home and for whom clinically assessed TUG times are available. In some cases, a P2TUG test result may be an estimate of a TUG test based on observing all the TUG test actions independently and in sub-sets throughout the daily activity of individuals. Estimation is accomplished through median times of actions as well as the median speed of walking. A linear regression is used to refine the estimated TUG action sequence time to P2TUG using a regression model trained on supervised data.

Linear regression may be used for its simplicity and interpretability of the data by clinicians. Alternative non-linear models can be used such as Random Forest Regression, convolutional neural net regression, and the like. While non-linear models could provide better estimates it may become more difficult to interpret the results by clinicians.

Trends of TUG and P2TUG times may be formed from the collected and analyzed data. A linear fit to TUG and P2TUG times is intended to provide a way to detect anomalies (for example, large deviations from linear fit) and as well as declines in mobility. In some cases, based on CDC recommendation, all time estimates that are above 12 sec are flagged.

In select embodiments, alternative privacy preserving sensors may be used in, without changing the core of the automated TUG detection or P2TUG estimation methods. A different type of radar may be used, or a Wi-Fi based sensor may be used. It is intended that the at least one sensor will not capture photo, video or other detailed scan of the individual. The use of a different type of radar or the use of Wi-Fi may result in different signal processing, for example WiFi Channel State Information (CSI), however the process of detecting TUG actions, detecting TUG and estimating P2TUG using TUG actions is not significantly affected. Alternatively, cameras may be used as sensors to detect TUG and P2TUG. In this case, detection of the actions may differ in the signal processing but once actions are detected the process of obtaining TUG and P2TUG is intended to be significantly similar.

In select embodiments, TUG action times may be combined into a single time then used in a regression model to estimate P2TUG. In alternative embodiments, TUG action times and other metrics such as speeds (including lay down and raise up from bed) may be used directly as a multidimensional vector into a regression model to estimate TUG.

In select embodiments, the systems and methods of the present disclosure may be operated in continuous passive and/or active modes. Operating in a continuous passive mode during the normal daily activity of the test subject is intended to allow for the TUG and/or P2TUG tests and results to be determined without a need for the subject to alter their habits. Operating in an active mobility testing scenario where the full TUG test can be self-administrated or administrated by a supervisor (e.g., caregiver or the like) allows for direct measurement of TUG in a subject's home.

In some cases, interactive administration using embodiments of the system identified herein are intended to be useful in a clinical setting and home setting where clinician or caregiver or the like is required to administer test to track rehabilitation or mobility improvement efforts. Further, active self-administrated may be beneficial where the person is cognitively able to remember and perform the TUG test (without assistance) but may prefer a less complicated option or may not have the technical understanding to use the interactive method via voice or smartphone interface. In a further case, a passive mode may be used. In some cases, the passive mode may be for trend tracking and determining when a person is becoming a fall risk. Although the results may be less accurate than the other modes, since it is an estimate, it is intended to flag when a person is at risk and should have further mobility assessed.

In select embodiments, privacy preserving sensors may be employed in the embodiments of the system and method of the present disclosure. Unlike video camera-based solutions, privacy preserving ambient sensors protect the privacy of the subjects, in particular when the sensors are operated continuously for passive measurement of P2TUG. In select embodiments, embodiments of the system and method of the present disclosure do not employ wearable sensors, and therefore do not rely on subjects wearing a sensor on themselves at all times unlike various conventional smartphone or IMU based approaches.

In select embodiments, the embodiments of the system and method of the present disclosure may employ a single sensor per room of a subject's house, which may make installation of the present systems easier. Furthermore, the systems and methods of the present disclosure may not require a calibration routine to calibrate sensor data to real-world coordinates. It will be understood that radar measures distances as such, may already provide world measurements in meters as a function of its settings. Conventional cameras, on the other hand, capture images and loses depth information unless calibrated for specific lenses used and position of the camera in the environment.

In select embodiments, the system may further be connected to and provide reports to an occupational therapist or physiotherapist. The occupational therapist or physiotherapist may review the trends received from the system and adjust their treatment regimen (for example, exercise frequency, exercise type, or the like) for the user of the system. In select embodiments the system may also suggest particular exercise regiments to boost a users' strength and endurance on the detection of declines.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether the embodiments or elements thereof described herein are implemented as a software routine, hardware circuit, firmware, or a combination thereof.

Embodiments of the disclosure or elements thereof can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

What is claimed is:

1. A system for ambient mobility testing comprising:
at least one sensor configured to collect data associated with an individual's movement;
an analysis module configured to analyze the collected data to determine a set of timed-up-and-go (TUG) actions of a TUG test and determine results of a complete TUG test; and
a reporting module configured to provide the results of the TUG test.

2. The system of claim 1 wherein, the at least one sensor is a privacy preserving sensor.

3. The system of claim 2 wherein, the at least one sensor is an mm-wave radar, LIDAR sensor or a WI-FI sensor.

4. The system of claim 1 further comprising: a monitoring module configured to monitor for TUG actions to update the results of the TUG test.

5. The system of claim 1, wherein the analysis module is further configured to determine a decline in mobility from the TUG actions.

6. The system of claim 1, wherein the analysis module is further configured to determine an abnormality within the results of the TUG test or within the TUG actions.

7. The system of claim 1, wherein the at least one sensor is configured to commence monitoring the individual's movement on a start command.

8. The system of claim 1, wherein the at least one sensor is configured to continuously monitor and the analysis module is configured to determine which collected data is associated with a TUG action.

9. The system of claim 1, wherein the analysis module is configured to aggregate a plurality of TUG actions performed during a predetermined period of time to determine a set of actions for the TUG test.

10. The system of claim 9 wherein the predetermined period of time may be between 1 hour and 1 week.

11. A method for ambient mobility testing, the method comprising:
collecting data associated with an individual's movement, via at least one sensor;
analyzing the collected data to determine timed-up-and-go (TUG) actions of a TUG test:
determining results of a complete TUG test; and
providing the results of the TUG test.

12. The method of claim 11 wherein the data is collected via a privacy preserving sensor.

13. The method of claim 12, wherein the least one sensor is an mm-wave radar, LIDAR sensor or a WI-FI sensor.

14. The method of claim 11, further comprising: monitoring for TUG actions to update the results of the TUG test.

15. The method of claim 11, further comprising: determining a decline in mobility from the TUG actions based on the results of the TUG test.

16. The method of claim 11 further comprising: determining an abnormality within the results of the TUG test or within the TUG actions.

17. The method of claim 11, wherein the collecting of data commences on a start command.

18. The method of claim 11 wherein the data may be continuously collected and analyzed to determine which collected data is associated with a TUG action.

19. The method of claim 11 further comprising: aggregating a plurality of TUG actions performed during a predetermined period of time to determine a set of actions for the TUG test.

20. The method of claim 19 wherein the predetermined period of time may be between 1 hour and 1 week.
